(19) United States
(12) Patent Application Publication
(10) Pub. No.: US 2025/0186421 A1
(43) Pub. Date: Jun. 12, 2025

(54) METHODS OF TREATING INFLAMMATORY AND/OR ALLERGIC DISORDERS WITH NORKETOTIFEN

(71) Applicant: Bridge Pharma, Inc., Lakewood Ranch, FL (US)

(72) Inventors: Gunnar Aberg, Lakewood Ranch, FL (US); Kresimir Pucaj, Toronto (CA)

(73) Assignee: Bridge Pharma, Inc., Lakewood Ranch, FL (US)

(21) Appl. No.: 19/061,157

(22) Filed: Feb. 24, 2025

(51) Int. Cl. A61K 31/4535; A61K 9/00; A61K 31/69; A61K 39/00; A61K 39/395; A61P 17/04

(52) U.S. Cl. CPC A61K 31/4535; A61K 9/0019; A61K 9/0053; A61K 31/69; A61K 39/3955; A61P 17/04; A61K 2039/505

(57) ABSTRACT

The methods disclosed herein relate to methods of orally administering norketotifen to human subjects. Oral norketotifen can be administered with food to reduce nausea and vomiting in the human subject. Oral norketotifen can be coadministered with additional medications such as dupilumab or crisaborole, or it can be administered after discontinuation of dupilumab or crisaborole.

### TECHNICAL FIELD

The embodiments disclosed herein relate to methods of treating inflammatory and/or allergic disorders such as atopic dermatitis, psoriasis, pruritus, asthma, and COPD with norketotifen.

### BACKGROUND

Inflammatory and/or allergic dermal disorders can be characterized by inflammation, redness, itching and/or flaking of skin. Such disorders include eczema, psoriasis, and atopic dermatitis, for example.

Atopic dermatitis (AD) is a chronic/relapsing inflammatory skin disease characterized by intense pruritus (e.g., severe itch) and by scaly and dry eczematous lesions. AD is often associated with other atopic disorders such as allergic rhinitis and asthma. Severe disease can be extremely disabling due to major psychological problems, significant sleep loss, and impaired quality of life, leading to high socioeconomic costs.

Topical corticosteroids (TCSs) are considered the mainstay of AD therapy and can be used for acute flares as well as for long-term use. Topical calcineurin inhibitors (TCIs) are a second-line option that are effective and well tolerated for continuous short-term use. Biologics like Dupixent® (dupilumab) are effective and popular but are disadvantaged by their extremely high cost and/or immunosuppressive effects. There is still a need for effective, safe, and less-expensive alternative treatment options due to concerns about the possibility of side effects, which may impact adherence and lead to inadequate effectiveness.

Pulmonary disorders such as asthma and chronic obstructive pulmonary disorder (COPD) are significant pulmonary problems in humans. Asthma is an inflammatory disease of the lungs that affects all age groups of patients and is characterized by recurrent attacks (exacerbations) of breathlessness and wheezing. The global prevalence of asthma is about 330 million patients with an annual asthma-related death rate of about 500,000 patients. Asthma-related deaths occur in connection with exacerbations of the disease. COPD is an umbrella term that is used to cover certain inflammatory lung diseases such as “emphysema” and “chronic bronchitis”. The estimated global prevalence of COPD is similar to asthma with about 330 million patients worldwide. However, the annual COPD-related death rate has been estimated at 3.2 million patients. COPD-related deaths occur in connection with exacerbations of the disease. There is also a need for effective and safe alternative treatment options for treating asthma and COPD.

What is needed are new treatment regimens for inflammatory and/or allergic dermal disorders such as atopic dermatitis and pulmonary disorders such as asthma and COPD.

### SUMMARY

In one aspect, a method of administering norketotifen (also referred to herein as NK) to a human subject comprises orally administering a dose of norketotifen to the human subject with food, wherein administering does not produce nausea or vomiting in the human subject.

In another aspect, a method of treating a human subject with an inflammatory dermal or pulmonary disorder comprises administering a subcutaneous dupilumab injection to the human subject, then discontinuing the subcutaneous dupilumab injection, and subsequent to discontinuing the subcutaneous dupilumab injection, orally administering norketotifen to the human subject, wherein the norketotifen is administered in a daily dosage amount of 2 mg/day to 64 mg/day.

In another aspect, a method of treating a human subject with an inflammatory dermal or pulmonary disorder comprises co-administering a subcutaneous dupilumab injection and oral norketotifen to the human subject, wherein the norketotifen is administered in a daily dosage amount of 2 mg/day to 64 mg/day.

In a further aspect, a method of treating a human subject with an inflammatory dermal or pulmonary disorder comprises determining subcutaneously administered dupilumab is contraindicated in the human subject, and, instead of administering subcutaneous dupilumab, orally administering norketotifen to the human subject, wherein the norketotifen is administered in a daily dosage amount of 2 mg/day to 64 mg/day, wherein dupilumab is not administered to the human subject.

### BRIEF DESCRIPTION OF THE DRAWINGS

FIG. 1 shows the incidence of vomiting in dogs administered norketotifen as a function of Cmax.

FIG. 2 shows the incidence of vomiting in dogs administered norketotifen as a function of AUC.

The above-described and other features will be appreciated and understood by those skilled in the art

### DETAILED DESCRIPTION

Described herein are treatment regimens for inflammatory and/or allergic dermal disorders such as atopic dermatitis, and pulmonary disorders such as asthma and COPD. The treatment regimens described herein are based on the results of animal studies and a Phase 2 clinical trial in which orally-administered norketotifen was compared to Eucrisa®, by dermal application, and Dupixent®, by subcutaneous administration. Eucrisa®, 2% crisaborole ointment, is a phosphodiesterase-4 inhibitor indicated for topical treatment of mild-to-moderate atopic dermatitis in adult and pediatric patients 3 months of age and older. Eucrisa® is applied topically twice daily to affected areas. The most common side effect of Eucrisa® is application site pain (burning or stinging sensation). Dupixent®, dupilumab injection, is an interleukin-4 and interleukin-13 receptor alpha antagonist indicated, for example, for treatment of patients with moderate-to-severe AD whose disease is not controlled with topical therapies or for whom such topical therapies are not advisable. Orally-administered norketotifen is shown to be as effective as Eucrisa® by dermal application, and Dupixent® by subcutaneous administration, and norketotifen provides an excellent, lower-cost alternative to these treatments.

As used herein, the term biophase refers to the site(s) where a drug expresses its therapeutic activity. It has previously been found that after oral administration, racemic norketotifen (RS-norketotifen) and the isomers thereof (R-norketotifen, S-norketotifen) accumulate in the skin. The skin is the biophase for dermal diseases and the pharmacokinetic exposure parameters-area under the curve (AUC), half-lives and mean residence times-of norketotifen in this biophase have been found to significantly exceed the corresponding parameters in the systemic circulation. For example, norketotifen has been shown to accumulate in the skin with a partition coefficient (AUC skin: AUC plasma) of 10.3, and norketotifen is associated with a very long terminal half-life (t1/2e) of 6-7 days. Thus, norketotifen can advantageously be used to treat dermal inflammatory and/or allergic diseases.

Norketotifen, after oral administration, is rapidly absorbed and is also preferentially distributed to the lungs, where the concentration of RS-norketotifen can reach concentrations that may be 100 times higher than the plasma concentration.

As used herein, the term norketotifen refers to norketotifen free base, an isomer, an isomeric mixture, a prodrug, or a pharmaceutically-acceptable salt thereof. RS-norketotifen refers to racemic norketotifen. In an aspect, the term norketotifen stands for RS-norketotifen hydrogen fumarate. Norketotifen is an achiral molecule, but has two atropisomers, S-norketotifen and R-norketotifen, as has previously been described by Aberg et al. in U.S. Pat. Nos. 7,226,934 and 7,557,128.

Norketotifen can be made using methods known in the art, as described in U.S. Pat. No. 3,682,930, the disclosure of which is hereby incorporated by reference for its teaching of the synthesis of norketotifen.

A prodrug of norketotifen is defined as a compound that after administration to a mammal can be converted to norketotifen. Prodrugs of norketotifen include N-substituted hydroxyalkyl or carboxyalkyloxyalkyl analogs thereof. Examples of such molecules are described in U.S. Pat. No. 6,207,683.

As used herein, the terms “pharmaceutically-acceptable salts” or “a pharmaceutically-acceptable salt thereof” refer to norketotifen salts, which have been prepared from pharmaceutically-acceptable non-toxic acids. Exemplary pharmaceutically-acceptable acids for the compound of the present invention include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrochloride salt and the hydrogen fumarate salt as well as the free base of norketotifen are particularly preferred.

In an aspect, norketotifen is administered at a daily dose of 2 mg to 64 mg, 2 mg to 32 mg, 2 mg to 16 mg or 2 mg to 8 mg. As shown in Example 1, based on the calculated margin of safety, a recommended maximum daily dosage of norketotifen is 2 mg/day to 64 mg/day.

In the Phase 2 study described herein, an initial loading dosage of 8 mg was used for the first 4 days (from the 8 mg/day for 7 days Phase 1 study, it was found that the plasma steady-state of norketotifen is reached within the first 4 days of dosing), after which patients received a dosage of 4 mg/day (maintenance dose) for the remaining 24 days of the study.

In an aspect, the oral dosage of norketotifen to a human patients suffering from dermal diseases may start with a daily oral loading dosage of from about 4 mg/day to about 64 mg/day of racemic norketotifen for 3 to 14 consecutive days, preferably four consecutive days, followed by a maintenance dosage of norketotifen of 2 mg/day to 32 mg/day mg. The maintenance dosage can be administered daily, or less frequently, such as every other day every three days, every 4 days, every 5 days, every 6 days, or once per week. The maintenance dosing will continue for as long as the human patient is suffering from the underlying disease.

In an aspect, a method of administering NK to a human subject in need of treatment for a dermal or pulmonary disorder comprises administering a daily dosage of NK that is greater than 40 mg/day but less than 64 mg/day, wherein the daily dosage of NK is within the margin of safety based on the no adverse event level for dogs and the human systemic exposure determined as area under the curve.

In all of the methods described herein, when oral norketotifen is used to treat an inflammatory dermal disorder, the methods may further comprise administering a topical corticosteroid.

### Administering Norketotifen With Food

In another aspect, norketotifen is administered on a full stomach. As shown in Example 2, administration of norketotifen with food to dogs provides a systemic Cₘₐₓ and AUC that are reduced about 37% and 26%, respectively, compared to animals where NK was administered on an empty stomach. This is an important result because the high Cmax in dogs was associated with nausea and vomiting.

In an aspect, a method of administering norketotifen to a human subject comprises orally administering norketotifen to the human subject, wherein the norketotifen is administered with food, and wherein administering does not produce nausea or vomiting in the human subject. The human subject may be suffering from an inflammatory dermal disorder such as atopic dermatitis, eczema or psoriasis, or an inflammatory pulmonary disorder such as asthma or COPD.

As used herein, administering with food means orally administering norketotifen with a meal, such as a meal that provides about 200 to about 1000 calories of energy. The norketotifen can be administered with a standard meal, a meal that provides about 200 to about 1000 calories of energy, a meal that provides about 400 to about 1000 calories of energy, a meal that provides about 500 to about 1000 calories of energy, a meal that provides about 50% of the calories derived from the fat, a meal that provides about 400 to about 700 calories of energy from fat, a meal that provides no fat or up to about 50 g of fat, a meal that provides 10 g up to about 50 g of fat, and the like.

In an aspect, a method of administering norketotifen to a human subject comprises orally administering a dose of norketotifen to the human subject with food, wherein administering does not produce nausea or vomiting in the human subject. In the Phase 2 study described herein, human subjects were dosed on a full stomach, and no nausea or vomiting was noted up to a dose of 16 mg.

In an aspect, the norketotifen is administered at a dose of 2 mg to 64 mg, 2 to 32 mg, 2 to 16 mg, or 2 to 8 mg.

### Norketotifen and Dupilumab

Dupilumab (marketed as Dupixent®) is a recombinant human IgG4monoclonal antibody that inhibits interleukin-4 (IL-4) and interleukin-13 (IL-13) signaling by specifically binding to the IL-4Rα subunit shared by the IL-4 and IL-13 receptor complexes which is indicated for the treatment of atopic dermatitis, asthma, chronic rhinosinusitis with nasal polyps, eosinophilic esophagitis, purigo nodularis, and COPD. Dupilumab is injected subcutaneously in amounts of 200 mg to 600 mg. In adults, dupilumab may be injected as a loading dose of 600 mg (two 300 mg injections) followed by one 300 mg injection every other week. Dosages may be adjusted for pediatric patients. In an aspect, the dupilumab subcutaneous injection is administered over at least 16 weeks, or at least 52 weeks.

A method of treating a human subject with an inflammatory dermal or pulmonary disorder comprises co-administering a subcutaneous dupilumab injection and oral norketotifen to the human subject, wherein the norketotifen is administered in a daily dosage amount of 2 mg/day to 64 mg/day. Administering both subcutaneous dupilumab injection and oral norketotifen can be beneficial to enhance the therapeutic effect as dupilumab inhibits Il-4 and Il-13 signaling, while NK inhibits release of Il-4 and Il-13 from pro-inflammatory cells, potentially allowing for lower doses of each individual drug and reducing the risk of side effect. In an aspect, the norketotifen is administered every day or less frequently.

As shown in the Phase 2a clinical trial results described herein, norketotifen was similarly efficacious in the treatment of atopic dermatitis to a subcutaneous dupilumab injection. Norketotifen is thus a viable option for patients who have discontinued dupilumab or for whom dupilumab is contraindicated.

As described in Kang et al. (Four-year long-term drug survival of dupilumab analyzed by treatment period and predictive factors of discontinuation in moderate to severe atopic dermatitis patients: A real-world retrospective study”, Journal of the American Academy of Dermatology, 91(3), supplement AB100, September 2024), almost 20% of patients receiving dupilumab for the treatment of atopic dermatitis discontinued treatment for reasons including adverse events, lack of efficacy, cost, and others.

In an aspect, a method of treating a human subject with an inflammatory dermal or pulmonary disorder comprises administering a subcutaneous dupilumab injection to the human subject, then discontinuing the subcutaneous dupilumab injection, and, subsequent to discontinuing the subcutaneous dupilumab injection, orally administering norketotifen to the human subject, wherein the norketotifen is administered in a daily dosage amount of 2 mg/day to 64 mg/day. In this aspect, once the dupilumab injection is discontinued, oral norketotifen administration is started, such that norketotifen and dupilumab are not coadministered.

In an aspect, the human subject has an inflammatory dermal disorder such as atopic dermatitis, psoriasis, or eczema. In an aspect, the human subject has slight, moderate, or severe atopic dermatitis.

In another aspect, the human subject has an inflammatory pulmonary disorder selected from asthma and COPD.

In an aspect, the oral norketotifen is administered with food.

In an aspect, the human subject discontinued dupilumab due to adverse events, dislike of needles, cost, access, inadequate response, lifestyle incompatibility, medication errors, drug interactions, pregnancy, or a combination thereof. Exemplary adverse events include conjunctivitis, blepharitis, oral herpes, eye pruritus, herpes simplex, and eosinophilia. Conjunctivitis is particularly observed in patients with atopic dermatitis treated with dupilumab.

Norketotifen is also useful in dupilumab non-responders, partial responders and non-durable responders. Real-world evidence has suggested that dupilumab is less effective than indicated by clinical trials (see, e.g., Bult et al., “Dupilumab responder types and predicting factors in patients with type 2 severe asthma: A real-world cohort study”, Respiratory Medicine, 231(2024) 107720; Papp et al., “Practical Management of Patients with Atopic Dermatitis on Dupilumab”, Dermatol Ther, 11(2021) 1805-1828). Dupilumab non-responders, that is, patients who are dupilumab-refractory, make up as much as 25% of patients treated with dupilumab. Partial responders exhibit some improvement with dupilumab, but do not meet the primary efficacy endpoint. Non-durable responders initially achieve therapeutic efficacy, but have a subsequent partial loss of efficacy.

Dupilumab has been shown to inhibit both Il-4 and Il-13 signaling. Norketotifen, in addition to inhibiting Il-4 and Il-13, also inhibits Il-31 (TH 2 cells play a central role in AD and release high levels of TH 2-associated cytokines including IL-31, thereby mediating inflammatory responses, initiating immunoregulatory circuits, stimulating itch; See Datsi, et al, “Interleukin-31: The “itchy” cytokine in inflammation and therapy, Allergy, 76(10), 2982-2997, 2001). Additionally, norketotifen is inhibiting eosinophil chemotaxis and has antimicrobial effects and can help prevent secondary S. aureus infections and the like. Because of these additional mechanisms of action for norketotifen compared to dupilumab, it is believed that norketotifen may rescue dupilumab non-responders, partial responders and/or non-durable responders.

Thus, in an aspect, the human subject discontinuing dupilumab is a dupilumab non-responder, partial responder, or non-durable responder.

In certain aspects, the subcutaneous dupilumab injection is administered to the human subject over at least 16 weeks prior to discontinuing.

In an aspect, the oral norketotifen can be co-administered with a topical corticosteroid.

In an aspect, a method of treating a dupilumab non-responder, partial responder or non-durable responder comprises co-administering a subcutaneous dupilumab injection and oral norketotifen to the subject, wherein the norketotifen is administered in a daily dosage amount of 2 mg/day to 64 mg/day. It is expected that co-administering dupilumab and norketotifen will improve the therapeutic response of the patient.

In another aspect, a method of treating a human subject with an inflammatory dermal or pulmonary disorder comprises determining subcutaneously administered dupilumab is contraindicated in the human subject, and, instead of subcutaneous dupilumab, orally administering norketotifen to the human subject, wherein the norketotifen is administered in a daily dosage amount of 2 mg/day to 64 mg/day, wherein dupilumab is not administered to the human subject.

Norketotifen is particularly useful for patients with mild, moderate, or severe AD for whom subcutaneous injection of dupilumab is contraindicated such as patients with a parasitic infection, patients with edema or inflammation at the injection site, patients with poor circulation at the injection site, patients with impaired skin integrity, patients with bleeding disorders, and patients who do not consent to subcutaneous injections.

Dupilumab and other biologic drugs are contraindicated in patients with parasitic infections such as nematode infections. Because norketotifen does not weaken the body's immune system, it is believed that norketotifen will not reduce the body's ability to fight such infections. And given that norketotifen has antiprotozoal effects, norketotifen may have positive effects to reduce such infections.

### Norketotifen and Crisaborole

As shown in the Phase 2a clinical trial results described herein, the efficacy of oral norketotifen is comparable to topical crisaborole. Crisaborole (Eucrisa®, Pfizer) is a topical prescription ointment for the treatment of AD. Crisaborole is a PDE4 inhibitor with efficacy in clinical trials in patients aged two and older with mild to moderate AD when used over a course of 28 days. Crisaborole was shown to reduce pruritus, severity of AD and other signs and symptoms of AD, although stinging and burning at the application site is a concern.

In an aspect, a method of treating a human subject with an inflammatory dermal disorder comprises topically administering crisaborole once or twice daily to the human subject, and orally administering norketotifen to the human subject, wherein the norketotifen is administered in a daily dosage amount of 2 mg/day to 64 mg/day. In an aspect, the inflammatory dermal disorder is atopic dermatitis, psoriasis or eczema. The atopic dermatitis can be slight, moderate or severe atopic dermatitis. In an aspect, the norketotifen is administered every day or less frequently.

In another aspect, a method of treating a human subject with atopic dermatitis comprises topically administering crisaborole once or twice daily to the human subject, discontinuing the topical crisaborole, and subsequently orally administering norketotifen to the human subject, wherein the norketotifen is administered in a daily dosage amount of 2 mg/day to 64 mg/day.

In an aspect, the topical crisaborole is administered for at least 28 days prior to discontinuing.

Reasons for discontinuing topical crisaborole can include adverse events and/or lack of demonstrated efficacy.

In an aspect, the topical crisaborole causes dermatitis, stinging, burning and/or infection at the application site.

In another aspect, the topical crisaborole does not provide an Investigator's Global Assessment score of clear or almost clear skin and/or does not provide a Severity of Pruritus Score reduction of at least one unit.

The embodiments disclosed herein also provide pharmaceutical compositions, which comprise the compound norketotifen, its isomers, and salts, formulated together with one or more pharmaceutically-acceptable carriers.

Pharmaceutical compositions for oral administration of solid dosage forms include capsules, granules, pills, powders and tablets. In solid dosage forms, the active compound may be mixed with one or more pharmaceutically-acceptable excipients or carriers (such as for example sodium citrate, dicalcium phosphate), fillers or extenders (such as for example starch, lactose, sucrose, glucose, mannitol, silicic acid), binders (such as for example carboxymethylcellulose, alginates, gelatin, polyvinylpyrrolidone, sucrose, acacia), humectants (such as for example glycerol), solution retarding agents (such as for example paraffin), disintegrating agents (such as for example agar-agar, calcium carbonate, starch, alginic acid, silicates, sodium carbonate), absorption accelerators (such as for example quaternary ammonium compounds), wetting agents (such as for example cetyl alcohol, glycerol monostearate), absorbents (such as for example kaolin, bentonite clay), lubricating agents (such as for example talc, calcium stearate, magnesium stearate, polyethylene glycols, sodium lauryl sulfate), and/or other excipients, such as for example buffering agents.

Solid forms of capsules, granules, pills, and tablets can have coatings and/or shells (such as for example enteric coatings) known in the art. The compositions may also be designed to release the active ingredient(s) in a certain part of the gastrointestinal tract or in a controlled release, slow-release or in a delayed-release manner. The active compound(s) can also be microencapsulated with one or more of the above-mentioned excipients or other suitable excipients.

Liquid dosage forms for oral administration may be preferred administration forms to children suffering from pruritus. Such formulations include for example pharmaceutically acceptable solutions, emulsions, solutions, suspensions, syrups and elixirs. The liquid dosage form may also contain excipients known to those skilled in the art of drug formulations, such as for example diluents (such as for example water, other solvents and solubilizing agents, and mixtures thereof), and emulsifiers (such as for example ethanol, isopropyl alcohol, ethyl carbonate, ethyl acetate, benzyl alcohol, benzyl benzoate, propylene glycol, butylene glycol, dimethyl formamide, oils, oleic acid, glycerol, polyethylene glycols, sorbitan fatty esters, and mixtures thereof. The oral compositions may also include other excipients as known to those skilled in the art.

The compositions described here can also include other drugs with antipruritic activity, as for example a corticosteroid. Due to the antipruritic activity of norketotifen or the isomers thereof, a beneficial steroid-sparing effect will be possible when treating patients suffering from various types of pruritic disorders.

The invention is further illustrated by the following non-limiting examples.

### Example 1. Dose Levels and Safety of Norketotifen

In the Phase 2 study described herein, doses of 4 mg (maintenance) and 8 mg (loading) were used, with very good effects and with no toxicity. The margin of safety (MOS) was calculated by dividing animal: human doses in mg/kg, or dividing animal systemic exposure (AUC) at no adverse effect level (NOAEL) (a 28-day dog study), with human systemic exposure AUC (from human Phase 1 study), or by dividing animal: to human dose expressed in m2 (body surface area—BSA), or by calculating HED (human equivalent dose). The results are provided in Table 1:

TABLE 1
MoS for norketotifen in humans

| | Margins of safety (animal: human) - based on: | | | |
|---|---|---|---|---|
| Dose | mg/kg | BSA | HED | Systemic exposure |
| (mg) | basis | (mg/m2) | (mg/kg) | (AUC_{0-inf.}) |
| 2^{a} | 260-fold | 141-fold | 145-fold | 127-fold |
| 4^{b} | 130-fold | 77-fold | 72-fold | 72-fold |
| 8^{c} | 65-fold | 35-fold | 36-fold | 29-fold |
| 16^{d} | 32-fold | 17-fold | 18-fold | 19-fold |
| 32^{e} | 16-fold | 9-fold | 9-fold | 6-fold |
| 64^{e} | 8-fold | 4-fold | 4.5-fold | 3-fold |

^{a}First-in-Human Dose
^{b}4 mg dose was a maintenance dose used in Phase 2 study (used for 24 days in Phase 2 study)
^{c}8 mg dose was a dose used as loading dose for the 1^{st} four days in a 28-day Phase 2 study (2 times the maintenance dose)
^{d}16 mg dose used in Phase 1 single ascending dose (SAD) study, that we believe it would still be tolerated in a future repeated dose human study
^{e}32 and 64 doses and the margins of safety were extrapolated dose and not previously used in humans

Based on these data, a maximum daily dose of norketotifen of 64 mg is expected to be well-tolerated and to produce minimal adverse events.

### Example 2: Reverse Food Effect of Norketotifen

Norketotifen was administered to dogs with and without food. For fed animals, canned food (one can (374 g) of Lamb Ground Dinner with Chicken & Rice per dog was weighed then offered for a period of 30 min prior to dosing at 4 mg/kg. After dosing, dry food (25% protein) was offered. For fasted animals, dogs were fasted overnight and until 4 h post dose and then offered dry food. The results are given in Table 2.

TABLE 2
Fed and fasted administration of norketotifen to dogs

| | Cₘₐₓ (ng/mL) | AUC_{0-∞} (ng*mL/hr) |
|---|---|---|
| Fed | 58 | 390 |
| Fast | 93 | 522 |
| % difference | 37% | 26% |

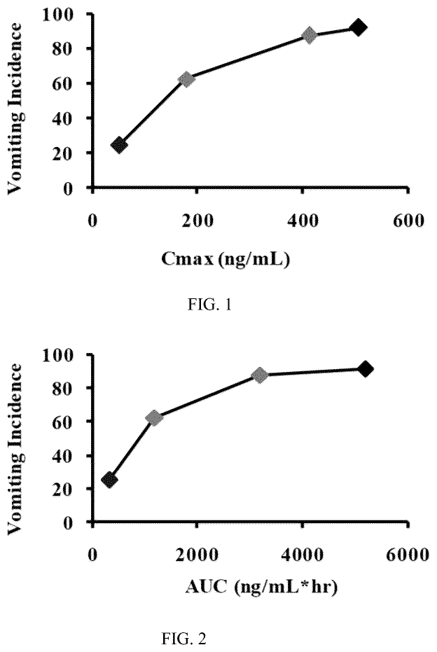

Cₘₐₓ and AUC were about 37% and 26% reduced, respectively, in fed animals compared to animals where norketotifen was administered on an empty stomach. This result is important since a high Cₘₐₓ norketotifen concentration in the plasma of dogs was associated with nausea and vomiting. For example, as shown in FIGS. 1 and 2, the incidence of vomiting increases as both systemic Cmax and AUC increase.

However, for the treatment of the dermal and pulmonary disorders described herein for treatment efficacy, the dermal/pulmonary exposure are more important than systemic exposure, that is, efficacy depends on the skin/pulmonary AUC/Cmax, which are considered to be sites of action for these conditions. The pharmacokinetic parameters in skin and plasma after oral dosing of norketotifen to dogs (N=5) are shown below.

TABLE 3
PK parameters of norketotifen in plasma
and skin of dogs after 28 days of dosing

| PK Parameter | NK Plasma | NK Skin |
|---|---|---|
| AUC_{0-∞} ng* h/mL | 4517 | 46,086 |
| t ½₍ₑ₎ (h)- | 38.3 | 25.4 |
| MRT (h) | 17.5 | 86.5 |
| t ½₍ₑ₎ (phase 2) (h) | — | 160 |
| MRT (phase 2)-(h) | — | 159 |

NK = RS-norketotifen;
t½ = half-life;
MRT = mean residence time
Phase 2 = 14 day post dosing period

By lowering the systemic exposure to norketotifen (e.g., administering with food), negative side effects can be minimized. Also, because a high norketotifen concentration in the biophase (skin or lungs) can be maintained, the therapeutic efficacy can be maintained.

### Example 3: Phase 2a Study

The efficacy and safety of norketotifen orally administered to human subjects over a 28 day period were determined in a Phase 2 study. Norketotifen efficacy was compared to the clinical data in the prescribing information for Eucrisa® by dermal application; and compared to the clinical data in the prescribing information (product monograph) for Dupixent® by subcutaneous administration.

In the Phase 2a study, 8 subjects were administered Placebo and 16 subjects were administered norketotifen. Oral norketotifen was administered at 4 mg BID (twice per day) for 4 days, followed by 4 mg QD (once per day). The duration of the study was 4 weeks. The parameters determined were:

IGA score, Investigator's Global Assessment scale. The scale is as follows:
0=clear—No inflammatory signs of atopic dermatitis (no erythema, no induration/papulation, no lichenification, no oozing/crusting). Post-inflammatory hyperpigmentation and/or hypopigmentation may be present.
1=almost clear—Barely perceptible erythema, barely perceptible induration/papulation, and/or minimal lichenification. No oozing or crusting.
2=mild—Slight but definite erythema (pink), slight but definite induration/papulation, and/or slight but definite lichenification. No oozing or crusting.
3=moderate—Clearly perceptible erythema (dull red), clearly perceptible induration/papulation, and/or clearly perceptible lichenification. Oozing and crusting may be present.
4=severe—Marked erythema (deep or bright red), marked induration/papulation, and/or marked lichenification. Disease is widespread in extent. Oozing or crusting may be present.

In the IGA assessment, a responder was identified as a subject with IGA 0 or 1 (clear or almost clear) after treatment. A partial responder was defined as having an IGA score>1, but less than the initial score recorded on Day 1. A non-responder is a subject in which the initial IGA score was identical to the IGA score at the end of treatment (i.e. no change in score numbers).

Pruritus: for percentage improvement for NK group
0: None
1: Mild (occasional, slight itching/scratching)
2: Moderate (Constant or intermittent itching/scratching/discomfort which is not disturbing sleep)
3: Severe (Bothersome itching/scratching/discomfort which is disturbing sleep)
   and NRS≥4 score improvement, percentage response for dupilumab group:
0: No pruritus
>0-<4: Mild pruritus
≥4-<7: Moderate pruritus
≥7-<9: Severe pruritus
≥9: Very severe pruritus

At the start of the study, the enrolled patients had an IGA assessment of 2 to 3 and a pruritus severity assessment of 1 to 3. In Table 4, the results for norketotifen were compared to the results from the Dupixent® prescribing information.

TABLE 4
Human efficacy and safety for norketotifen compared
to Dupixent ® in the treatment of AD

| | | | Dupilumab SOLO 1 Study | |
|---|---|---|---|---|
| | | | as Reported in Sanofi | |
| | NK Phase 2 Human | | Biotechnology Product | |
| | Trial | | Monograph for Dupixent ® | |
| | Placebo | NK | Placebo | Dupilumab |
| Dose | 4 mg BID* for 4 | | 300 mg Q2W* | |
| | days, followed by | | | |
| | 4 mg QD* for 24 | | | |
| | days | | | |
| Study Duration | 4 | 4 | 16 | 16 |
| (weeks) | | | | |
| Number of | 8 | 16 | 224 | 224 |
| Subjects | | | | |
| IGA scores ≤1, | 0% | 43.89% ^{b} | 10.3% | 37.9% |
| percent | | | | |
| responders ^{a} | | | | |
| IGA scores >1, | 50% | 50% | — | — |
| but less than the | | | | |
| initial scores on | | | | |
| Day 1 (partial | | | | |
| responders) | | | | |
| Non responders | 50% | 6.1% | — | — |
| at all | | | | |
| Pruritus, score | 12.5% | 62.5%^{c} | 12.3% | 40.8% |
| 0, percentage | | | | |
| improvement for | | | | |
| NK group; and | | | | |
| NRS ≥4 score | | | | |
| improvement, | | | | |
| percentage | | | | |
| response for | | | | |
| dupilumab | | | | |
| group | | | | |
| % reduction of | −36.8% | −63.3% | — | — |
| affected BSA | | | | |
| (Baseline to Day | | | | |
| 29) | | | | |

— not known
^{a}responder was defined as a subject with IGA 0 or 1 (clear or almost clear)
^{b}statistically different from Placebo at p = 0.0095 (Fisher's exact test) when only full responders were taken into account, and p = 0.0031 (Fisher's exact test) when partial responders and non-responders were included.
^{c} significantly different from Placebo at p = 0.016, and significantly decreased pruritus within the test group (initial to the end of treatment; Day 1 to Day 29) at p ≤ 0.001 (paired t-test).

As shown in Table 4, the efficacy between NK and dupilumab was very similar in respect to the % responders as judged by IGA scores of ≤1 (improvement between initial Day 1 scores and the last day of study scores). One caveat, as mentioned previously, is that in the real world, the percentage of responders to dupilumab appears to be smaller than indicated in the clinical studies. The p-value of the Fisher's exact test was 0.0031<0.05, suggesting that NK (Test) was likely associated with higher success rates in IGA outcomes compared to the Placebo. The p-value indicated a statistically-significant association between the treatment and IGA success rates, assuming a 5% significance level (α=0.05).

Of note, the results for norketotifen were achieved within 28 days (4 weeks) of treatment, while the dupilumab results were after a 16-week period. Without being held to theory it is believed that results for norketotifen could exceed those for dupilumab after a 16-week treatment period.

An analysis of the safety population indicates that norketotifen was well-tolerated, with no reported instances of serious adverse events or treatment-related adverse events. In conclusion, given the absence of serious adverse events, and the occurrence of an adverse event incidence in only one patient that resolved completely without any sequelae, the safety profile of norketotifen may be considered as acceptable for the treatment of atopic dermatitis.

It is important to note that the comparison between the effects of the treatment of AD used in Table 4 is an approximate comparison for the following reasons: subjects receiving norketotifen were dosed QD for 28 days, while subjects receiving dupilumab were dosed Q2W for 16 weeks; the scoring systems used for assessment of pruritus were different (NK study used a scale based on severity scores ranging from 0 to 3, where 0 is no pruritus and 3 is severe pruritus; while the dupilumab study used an NRS score ranging from 0 to 10, where 0 is no pruritus and 10 is worst itch imaginable); and the subject population in the norketotifen study included individuals with slight to moderate AD, while the subject population in the dupilumab study also included individuals with severe AD.

In Table 5, the results for norketotifen were compared to the results from the Eucrisa® prescribing information.

TABLE 5
Human efficacy and safety for norketotifen compared
to Eucrisa ® in the treatment of AD

| | | | EUCRISA (crisaborole) | |
|---|---|---|---|---|
| | | | Study AN2728-AD-302 | |
| | | | per Pfizer product | |
| | NK Phase 2a Human Trial | | monograph | |
| | Placebo | NK | Placebo | EUCRISA |
| Number of | 8 | 16 | 250 | 513 |
| Subjects | | | | |
| IGA* scores ≤1, | 0% | 43.89% ^{b} | 29.7% | 48.5% |
| percent | | | | |
| responders ^{a} | | | | |

| | | |
|---|---|---|
| p-value | 0.0031 | <0.001 |

*IGA = Investigator's Global Assessment scale
^{a} responder was defined as a subject with IGA 0 or 1 (clear or almost clear)
^{b} statistically different from Placebo at p = 0.0095 (Fisher's exact test) when only full responders were taken into account, and p = 0.0031 (Fisher's exact test) when partial responders and non-responders were included.

As shown in Table 5, orally-administered norketotifen was at least as effective as topically-administered crisaborole.

### EMBODIMENTS

Embodiment 1: A method of administering norketotifen to a human subject, comprising orally administering a dose of norketotifen to the human subject with food, wherein administering does not produce nausea or vomiting in the human subject.
Embodiment 2: The method of embodiment 1, wherein a daily dosage of norketotifen is 2 mg to 64 mg.
Embodiment 3: The method of embodiment 1, wherein a daily dosage of norketotifen is 2 mg to 32 mg.
Embodiment 4: The method of embodiment 1, wherein a daily dosage of norketotifen is 2 mg to 16 mg.
Embodiment 5: The method of embodiment 1, wherein a daily dosage of norketotifen is 2 mg to 8 mg.
Embodiment 6: The method of embodiment 1, wherein the human subject has an inflammatory dermal disorder.
Embodiment 7: The method of embodiment 6, wherein the inflammatory dermal disorder is atopic dermatitis, psoriasis or eczema.
Embodiment 8: The method of embodiment 1, wherein the human subject has an inflammatory pulmonary disorder selected from asthma and COPD.
Embodiment 9: The method of embodiment 1, wherein the norketotifen is in the form of RS-norketotifen, R-norketotifen, S-norketotifen, or a pharmaceutically acceptable salt thereof.
Embodiment 10: The method of embodiment 6, further comprising administering a topical corticosteroid.
Embodiment 11: A method of treating a human subject with an inflammatory dermal or pulmonary disorder, comprising administering a subcutaneous dupilumab injection to the human subject; then discontinuing the subcutaneous dupilumab injection; and subsequent to discontinuing the subcutaneous dupilumab injection, orally administering norketotifen to the human subject, wherein the norketotifen is administered in a daily dosage amount of 2 mg/day to 64 mg/day.
Embodiment 12: The method of embodiment 11, wherein the human subject has an inflammatory dermal disorder.
Embodiment 13: The method of embodiment 12, wherein the inflammatory dermal disorder is atopic dermatitis, psoriasis or eczema.
Embodiment 14: The method of embodiment 13, wherein the human subject has slight, moderate or severe atopic dermatitis.
Embodiment 15: The method of embodiment 11, wherein the human subject has an inflammatory pulmonary disorder selected from asthma and COPD.
Embodiment 16: The method of embodiment 11, wherein the human subject discontinued dupilumab due to an adverse event, dislike of needles, cost, access, inadequate response, lifestyle incompatibility, medication errors, drug interactions, pregnancy, or a combination thereof.
Embodiment 17: The method of embodiment 16, wherein the adverse event is conjunctivitis, blepharitis, oral herpes, eye pruritus, herpes simplex, eosinophilia, or a combination thereof.
Embodiment 18: The method of embodiment 11, wherein the human subject is a dupilumab non-responder, partial responder, or non-durable responder.
Embodiment 19: The method of embodiment 11, wherein the subcutaneous dupilumab injection is administered to the human subject over at least 16 weeks prior to discontinuing.
Embodiment 20: The method of embodiment 12, further comprising administering a topical corticosteroid.
Embodiment 21: A method of treating a human subject with an inflammatory dermal or pulmonary disorder, comprising co-administering a subcutaneous dupilumab injection and oral norketotifen to the human subject, wherein the norketotifen is administered in a daily dosage amount of 2 mg/day to 64 mg/day.
Embodiment 22: The method of embodiment 21, wherein the human subject has an inflammatory dermal disorder.
Embodiment 23: The method of embodiment 22, wherein the inflammatory dermal disorder is atopic dermatitis, psoriasis or eczema.
Embodiment 24: The method of embodiment 23, wherein the human subject has slight, moderate or severe atopic dermatitis.
Embodiment 25: The method of embodiment 21, wherein the human subject has an inflammatory pulmonary disorder selected from asthma and COPD.
Embodiment 26: The method of embodiment 22, further comprising administering a topical corticosteroid.
Embodiment 27: The method of embodiment 21, wherein the norketotifen is administered every other day, every three days, every 4 days, every 5 days, every 6 days, or once per week.
Embodiment 28: A method of treating a human subject with an inflammatory dermal or pulmonary disorder, comprising determining subcutaneously administered dupilumab is contraindicated in the human subject; and, instead of administering subcutaneous dupilumab, orally administering norketotifen to the human subject, wherein the norketotifen is administered in a daily dosage amount of 2 mg/day to 64 mg/day, wherein dupilumab is not administered to the human subject.
Embodiment 29: The method of embodiment 28, wherein the human subject has an inflammatory dermal disorder.
Embodiment 30: The method of embodiment 29, wherein the inflammatory dermal disorder is atopic dermatitis, psoriasis or eczema.
Embodiment 31: The method of embodiment 30, wherein the human subject has slight, moderate or severe atopic dermatitis.
Embodiment 32: The method of embodiment 28, wherein the human subject has an inflammatory pulmonary disorder selected from asthma and COPD.
Embodiment 33: The method of embodiment 29, further comprising administering a topical corticosteroid.
Embodiment 34: The method of embodiment 28, wherein the human subject for whom dupilumab is contraindicated has a parasitic infection, edema or inflammation at the injection site, poor circulation at the injection site, impaired skin integrity, a bleeding disorder, does not consent to subcutaneous injections, or a combination thereof.
Embodiment 35: A method of treating a human subject with an inflammatory dermal disorder, comprising topically administering crisaborole once or twice daily to the human subject; and orally administering norketotifen to the human subject, wherein the norketotifen is administered in a daily dosage amount of 2 mg/day to 64 mg/day.
Embodiment 36: The method of embodiment 35, wherein the inflammatory dermal disorder is atopic dermatitis, psoriasis or eczema.
Embodiment 37: The method of embodiment 36, wherein the human subject has slight, moderate or severe atopic dermatitis.
Embodiment 38: The method of embodiment 35, wherein the norketotifen is administered every other day, every three days, every 4 days, every 5 days, every 6 days, or once per week.
Embodiment 39: A method of treating a human subject with atopic dermatitis, comprising topically administering crisaborole once or twice daily to the human subject; then discontinuing the topical crisaborole; and orally administering norketotifen to the human subject, wherein the norketotifen is administered in a daily dosage amount of 2 mg/day to 64 mg/day.
Embodiment 40: The method of embodiment 39, wherein the topical crisaborole is administered for at least 28 days prior to discontinuing the topical crisaborole.
Embodiment 41: The method of embodiment 39, wherein the topical crisaborole causes dermatitis, stinging, burning and/or infection at the application site.
Embodiment 42: The method of embodiment 39, wherein the topical crisaborole does not provide an Investigator's Global Assessment score of clear or almost clear skin and/or does not provide a Severity of Pruritus Score reduction of at least one unit.

The use of the terms “a” and “an” and “the” and similar referents (especially in the context of the following claims) are to be construed to cover both the singular and the plural, unless otherwise indicated herein or clearly contradicted by context. The terms first, second, etc., as used herein, are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to a preferred embodiment, it will be understood by those skilled in the art that various changes may be made and equivalents may be substituted for elements thereof, without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A method of administering norketotifen to a human subject, comprising orally administering a dose of norketotifen to the human subject with food, wherein administering does not produce nausea or vomiting in the human subject.

2. The method of claim 1, wherein a daily dosage of norketotifen is 2 mg to 64 mg.

3. The method of claim 1, wherein a daily dosage of norketotifen is 2 mg to 32 mg.

4. The method of claim 1, wherein a daily dosage of norketotifen is 2 mg to 16 mg.

5. The method of claim 1, wherein a daily dosage of norketotifen is 2 mg to 8 mg.

6. The method of claim 1, wherein the human subject has an inflammatory dermal disorder.

7. The method of claim 6, wherein the inflammatory dermal disorder is atopic dermatitis, psoriasis or eczema.

8. The method of claim 1, wherein the human subject has an inflammatory pulmonary disorder selected from asthma and COPD.

9. The method of claim 1, wherein the norketotifen is in the form of RS-norketotifen, R-norketotifen, S-norketotifen, or a pharmaceutically acceptable salt thereof.

10. The method of claim 6, further comprising administering a topical corticosteroid.

11. A method of treating a human subject with an inflammatory dermal or pulmonary disorder, comprising
administering a subcutaneous dupilumab injection to the human subject, then discontinuing the subcutaneous dupilumab injection, and
subsequent to discontinuing the subcutaneous dupilumab injection, orally administering norketotifen to the human subject, wherein the norketotifen is administered in a daily dosage amount of 2 mg/day to 64 mg/day.

12. The method of claim 11, wherein the human subject has an inflammatory dermal disorder.

13. The method of claim 12, wherein the inflammatory dermal disorder is atopic dermatitis, psoriasis or eczema.

14. The method of claim 13, wherein the human subject has slight, moderate or severe atopic dermatitis.

15. The method of claim 11, wherein the human subject has an inflammatory pulmonary disorder selected from asthma and COPD.

16. The method of claim 11, wherein the human subject discontinued dupilumab due to an adverse event, dislike of needles, cost, access, inadequate response, lifestyle incompatibility, medication errors, drug interactions, pregnancy, or a combination thereof.

17. The method of claim 16, wherein the adverse event is conjunctivitis, blepharitis, oral herpes, eye pruritus, herpes simplex, eosinophilia, or a combination thereof.

18. The method of claim 11, wherein the human subject is a dupilumab non-responder, partial responder, or non-durable responder.

19. The method of claim 11, wherein the subcutaneous dupilumab injection is administered to the human subject over at least 16 weeks prior to discontinuing.

20. The method of claim 12, further comprising administering a topical corticosteroid.

21. A method of treating a human subject with an inflammatory dermal or pulmonary disorder, comprising co-administering a subcutaneous dupilumab injection and oral norketotifen to the human subject, wherein the norketotifen is administered in a daily dosage amount of 2 mg/day to 64 mg/day.

22. The method of claim 21, wherein the human subject has an inflammatory dermal disorder.

23. The method of claim 22, wherein the inflammatory dermal disorder is atopic dermatitis, psoriasis or eczema.

24. The method of claim 23, wherein the human subject has slight, moderate or severe atopic dermatitis.

25. The method of claim 21, wherein the human subject has an inflammatory pulmonary disorder selected from asthma and COPD.

26. The method of claim 22, further comprising administering a topical corticosteroid.

27. The method of claim 21, wherein the norketotifen is administered every other day, every three days, every 4 days, every 5 days, every 6 days, or once per week.

28. A method of treating a human subject with an inflammatory dermal or pulmonary disorder, comprising determining subcutaneously administered dupilumab is contraindicated in the human subject, and, instead of administering subcutaneous dupilumab, orally administering norketotifen to the human subject, wherein the norketotifen is administered in a daily dosage amount of 2 mg/day to 64 mg/day, wherein dupilumab is not administered to the human subject.

29. The method of claim 28, wherein the human subject has an inflammatory dermal disorder.

30. The method of claim 29, wherein the inflammatory dermal disorder is atopic dermatitis, psoriasis or eczema.

31. The method of claim 30, wherein the human subject has slight, moderate or severe atopic dermatitis.

32. The method of claim 28, wherein the human subject has an inflammatory pulmonary disorder selected from asthma and COPD.

33. The method of claim 29, further comprising administering a topical corticosteroid.

34. The method of claim 28, wherein the human subject for whom dupilumab is contraindicated has a parasitic infection, edema or inflammation at the injection site, poor circulation at the injection site, impaired skin integrity, a bleeding disorder, does not consent to subcutaneous injections, or a combination thereof.

35. A method of treating a human subject with an inflammatory dermal disorder, comprising
topically administering crisaborole once or twice daily to the human subject, and
orally administering norketotifen to the human subject, wherein the norketotifen is administered in a daily dosage amount of 2 mg/day to 64 mg/day.

36. The method of claim 35, wherein the inflammatory dermal disorder is atopic dermatitis, psoriasis or eczema.

37. The method of claim 36, wherein the human subject has slight, moderate or severe atopic dermatitis.

38. The method of claim 35, wherein the norketotifen is administered every other day, every three days, every 4 days, every 5 days, every 6 days, or once per week.

39. A method of treating a human subject with atopic dermatitis, comprising
topically administering crisaborole once or twice daily to the human subject, then discontinuing the topical crisaborole, and
orally administering norketotifen to the human subject, wherein the norketotifen is administered in a daily dosage amount of 2 mg/day to 64 mg/day.

40. The method of claim 39, wherein the topical crisaborole is administered for at least 28 days prior to discontinuing the topical crisaborole.

41. The method of claim 39, wherein the topical crisaborole causes dermatitis, stinging, burning and/or infection at the application site.

42. The method of claim 39, wherein the topical crisaborole does not provide an Investigator's Global Assessment score of clear or almost clear skin and/or does not provide a Severity of Pruritus Score reduction of at least one unit.
